(19) United States
(12) Patent Application Publication
(10) Pub. No.: US 2023/0144385 A1
(43) Pub. Date: May 11, 2023

(54) ETHANOLAMINE FORMULATION FOR TREATING EPITHELIAL OVARIAN CARCINOMA

(71) Applicant: METANOI THERAPEUTICS, INC., Atlanta, GA (US)

(72) Inventor: Guilherme H.C. CANTUARIA, Atlanta, GA (US)

(21) Appl. No.: 17/995,341

(22) PCT Filed: Mar. 5, 2021
(86) PCT No.: PCT/US21/21007

§ 371 Date: Oct. 3, 2022

Related U.S. Application Data
(60) Provisional application No. 63/006,426, filed on Apr. 7, 2020.

(51) Int. Cl. A61K 31/133; A61P 35/00; C07K 16/28

(52) U.S. Cl. CPC A61K 31/133; A61P 35/00; C07K 16/2818

(57) ABSTRACT

Monoethanolamine (Etn) displays strong in vitro and in vivo efficacy in prostate cancer cell lines and xenograft models, respectively, as well as in cell lines from diverse cancer types. Etn is a pro-drug, which upon entry into tumor cells, is converted into cytotoxic phosphoethanolamine (PhosE). Etn treatment potently down-regulates HIF-1α and drives a catastrophic uncoupling of multiple pathways to induce metabolic crisis and cell death, selectively in tumor cells, while sparing normal cells. Importantly, the ovarian cancer cell line OVCAR3 was more sensitive to Etn than all the prostate, breast, colon, and pancreatic cancer cell lines tested. An Etn-based formulation with favorable pharmacokinetics/pharmacodynamics (PK/PD) can therefore in some embodiments be used as single therapeutic for EOC or OCCC.

### CROSS-REFERENCE TO RELATED APPLICATIONS

This application claims benefit of U.S. Provisional Application No. 63/006,426, filed April 7, 2020, which is hereby incorporated herein by reference in its entirety.

### BACKGROUND

Epithelial ovarian cancer (EOC) is a life-threatening disease characterized by late-stage presentation; EOCs are therefore a leading cause of death for gynecological cancers. The standard treatment for EOCs is debulking surgery followed by platinum-based chemotherapy. While these treatments are often initially efficacious, most patients develop recurrent disease, a largely incurable state. Ovarian clear cell carcinomas (OCCCs), a subtype of EOCs, are characterized by clear cells with aberrant lipid and glycogen accumulation. OCCC comprises 5-10% of ovarian carcinomas in North America, and ˜25% of EOCs in Japan. It frequently presents in perimenopausal women, and is often associated with endometriosis, thromboembolic vascular complications, and hypercalcemia. In contrast to high grade serous ovarian carcinoma, OCCC is usually detected in an early stage (stage I). Nonetheless, advanced stage/recurrent patients with OCCC have a much poorer prognosis than patients with other EOC subtypes mainly because the former are refractory to platinum-based regimens. Hence, there is an urgent unmet need for new OCCC treatment paradigms.

### SUMMARY

The standard treatment for ovarian clear cell carcinoma (OCCC), which comprises 10-15% of epithelial ovarian carcinomas (EOCs) in North America, is debulking surgery, followed by platinum-based chemotherapy. OCCCs are notoriously hard-to-treat as they are resistant to platinum-based chemotherapy; thus, OCCC patients have a worse prognosis, stage for stage, than patients with other EOC subtypes. There is an urgent unmet need to find new and effective treatments for OCCC. Malignant cells undergo metabolic reprogramming in response to tumor microenvironment (TME) stressors. Intratumoral hypoxia makes the TME immunosuppressive through its effects on both tumor cells and tumor-infiltrating immune cells. OCCCs express high levels of hypoxia-inducible factor-lalpha (HIF-1α), which reprograms cellular metabolism in response to hypoxia and activates genes promoting therapy resistance and cell survival. OCCC cells display aberrant lipid and glycogen accumulation—a sign of significantly reprogrammed metabolism. Monotherapy with immune checkpoint inhibitors (ICIs) has so far yielded disappointing results in ovarian cancer, and multiple trials are underway combining ICIs with drugs affecting other targets. Two immunotherapy studies from 2015 demonstrated responses in the small numbers of OCCC patients enrolled. OCCC and renal cell carcinomas (RCCs) share similar gene expression profiles and currently, Nivolumab, an ICI, is FDA-approved for RCC; thus, Nivolumab may merit further exploration in OCCC. Drugs that target metabolic vulnerabilities may synergize with Nivolumab to offer a more efficacious therapy for OCCC.

Monoethanolamine (Etn) is a pro-drug, which upon entry into tumor cells, is converted into cytotoxic phosphoethanolamine (PhosE). Etn treatment potently downregulates HIF-1α and drives a catastrophic uncoupling of multiple pathways to induce metabolic crisis and cell death, selectively in tumor cells, while sparing normal cells. Importantly, the ovarian cancer cell line OVCAR3 was more sensitive to Etn than all the prostate, breast, colon, and pancreatic cancer cell lines tested. An Etn-based formulation with favorable pharmacokinetics/pharmacodynamics (PK/PD) can therefore in some embodiments be used as single therapeutic for an EOC.

Therefore, disclosed herein is a method for treating an epithelial ovarian carcinoma (EOC) that involves administering to a subject in need thereof, an effective amount of a first pharmaceutical composition comprising monoethanolamine or a pharmaceutically acceptable salt thereof. and a pharmaceutically effective carrier. In some embodiments, the EOC comprises ovarian clear cell carcinoma (OCCC). In some embodiments, the EOC comprises serous ovarian carcinoma. In some embodiments, the EOC comprises endometrioid ovarian cancer. In some embodiments, the EOC comprises mucinous ovarian cancer.

The disclosed Etn compositions can in some embodiments be used as an adjuvant for a checkpoint inhibitor. In some embodiments, monoethanolamine is the only therapeutically active agent in the first pharmaceutical composition. In some embodiments, the pharmaceutical composition comprises monoethanolamine and a checkpoint inhibitor.

The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (M DX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. (A) Representative dose—response curve for Etn and PhosE on the proliferation of PC-3 cells (i). Percentage cell survival was measured by MTT assay after treating cells with increasing concentrations of Etn and PhosE for 48 hours at pH 7.4. Bar graph representation and photograph of crystal violet-stained surviving colonies from the control, Etn and PhosE-treated groups (ii). For clonogenic survival assay, PC-3 cells treated with 2 mg/mL Etn/PhosE at pH 7.4. (B) Antiproliferative effect of Etn treatment on prostate cancer cell lines (PC-3, DU145 and C42B) and normal cell line (RWPE-1). PC-3, DU145, C42B andRWPE-1 cells were treated with 0.5 and 1 mg/mL Etn for 48 hours at pH 7.4 followed by measurement of cell survival by MTT assay (i). IC50 values of Etn treatment of cancer cell lines MDA-MB-486 (breast), OVCAR-3 (ovarian), CFPAC (pancreatic)and PC-3 (ii).

FIG. 2. (A) Intracellular levels of Etn and PhosE upon treatment of PC-3 cells with Etn and PhosE. (B) Effect of choline kinase inhibition on proliferation of PC-3 cells. (C) Intracellular PhosE level upon Etn treatment.

FIG. 3. (Ai) Representative bioluminescent images of one animal per group indicating progression of tumor growth over 4 weeks in control and Etn-treated mice. (Aii) Tumor growth monitored (by vernier calipers) over a period of 4 weeks. (Aiii) Weight of tumors from control and Etn-treated mice. (B) Body weight of vehicle and Etn fed mice over a period of 4 weeks of treatment. (C) Intratumoral levels of PhosE and Etn in vehicle and Etn-fed mice after 4 weeks of Etn treatment.

FIG. 4. (A) Immunoblots of control and Etn-treated cell lysates for pRb, cdk4, cdk2, p21, c-PARP, Bim, Bcl-2 and β actin. (B) Effect of Etn treatment on annexin V binding to PC-3 cells. (C) Immunoblots of control and Etn-treated tumors lysates for p53, p21, Bax, pBcl-2, c-PARP, Bim, Bid and β actin. (D) Micrographs showing IHC staining of Ki67 and c-PARP in control and Etn-treated prostate cancer xenografts.

FIG. 5. (A) Immunoblots of control and Etn-treated cell lysates for HIF1-α. (B) Effect of Etn treatment on oxygen consumption rate in PC-3 cells. Intracellular glucose (Ci) and glutamine (Cii) levels in control and Etn-treated tumors. (D) Effect of choline kinase inhibition on intracellular levels of glucose (Di) and glutamine (Dii) in Etn-treated cells.

FIG. 6. (A) Representative TEMs of control and 40 mg/kg Etn-treated tumors showing changes in mitochondrial morphology and accumulation of lipids upon Etn treatment. Ultra-thin sections were cut on Boeckeler MTx ultramicrotome, counterstained with lead citrate, and examined on a LEO 906e TEM. Mitochondria and accumulated lipid granules are highlighted by red arrows. Treated tumors showed elongated mitochondria with degrading mitochondrial matrices (ii) and abundant lipid rich granules (iv) in comparison with control tumors (i and iii). Left panels, scale bar ¼2 mm; right panels, scale bar ¼5 mm. (B) Etn treatment increases lipid levels in Etn-treated tumors. Levels of PE (i), PS (ii), PC (iii), and SM (iv) lipids in control and Etn-treated tumors. In the abbreviation of lipid, 1st and 2nd numbers denote the number of carbon atoms and unsaturated bonds present in the lipid, respectively. Lipid amounts were quantified by LC/MS-MS. Values and error bars shown represent mean and SE, respectively.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The Kennedy pathway includes two parallel branches, one for phosphatidyl ethanolamine (PE) synthesis and the other for phosphatidylcholine (PC) synthesis. The PE synthesis pathway consists of three enzymatic steps, Ethanolamine kinase (EtnK) catalyzes the ATP-dependent phosphorylation of ethanolamine to form PhosE and ADP. ETnK is specific for ethanolamine; it does not catalyze the phosphorylation of choline. In the second, rate-limiting step, a CTP: phosphoethanolamine cytidyltrnnsferase (ECT) uses PhosE and CTP to form the high-energy donor CDP-ethanolamine with the release of pyrosphosphate. CDP-ethanolamine: 1,2-diacylglycerol ethanolaminephosphotransferase (EPT) catalyzes the final step in the pathway, using CDP-ethanolamine and a lipid anchor, such as diacylglycerol (DAG) or alky!-acylglycerol (AAG) to form PE and CMP.

The analogous pathway for PC synthesis uses a series of similar reactions, except for the involvement of choline instead of ethanolamine to form PC. However, in contrast to the PE pathway, the PC pathway includes several mammalian choline kinase (CK) isoforms with a choline/ethanolamine kinase (ChoK/EtnK) domain: ChoKα1 (NP_001268), ChoKα2 (NP_997634) and ChoKβ1 (NP_005189) that are able to phosphorylate both choline and ethanolamine. Previous studies suggest that ChoK acts as a dimeric protein forming different homo- or hetero-dirner isoform combinations resulting in different levels of ChoK activity, whereby the a/a homodimer Is the most active choline kinase form, the β/β homodirner the least active, and the α/β heterodimer has an intermediate phenotype.

One aspect of the present application relates to a method for treating cancer, comprising orally administering to a subject in need thereof, an effective amount of a pharmaceutical composition comprising Etn, or a pharmaceutically acceptable salt thereof, and a pharmaceutically effective carrier.

The Etn used in the treatment methods of the present disclosure may be isolated and purified from a natural product or a processed product thereof, or a synthesized product. Ethanolamine can be produced by reacting ethylene oxide and ammonia. Ethanolamine can also be isolated and purified from a natural product or a processed product thereof by known techniques such as solvent extraction, various chromatographic methodologies and the like, Alternatively, ethanolamine may be obtained from commercial sources, for example, Sigma-Aldrich Co., Ltd. and the like.

In other embodiments, the method of treating cancer comprises administering to a subject in need thereof, an effective amount of a pharmaceutical composition comprising an analog of Etn, a prodrug of Etn, an Etn hybrid molecule or a pharmaceutically acceptable salt thereof; and a pharmaceutically effective carrier. In certain embodiments; the pharmaceutical compositions may further include one or more additional anticancer agents. Exemplary anticancer agents include anti-mitotic agents, anti-interphase agents, anti-microtubule agents, anthracycline-based agents, aromatase inhibitor agents, anti-angiogenesis agents, immune checkpoint regulators, and combinations thereof.

In some embodiments, the pharmaceutical composition is administered by oral, intravenous, intraperitoneal, subcutaneous, intranasal or dermal administration. In some embodiment, wherein the pharmaceutical composition is administered as a solid or semi-solid in capsules.

In certain embodiments, the Etn analog is a compound represented by the following formula: X—CH₂—CH₂—O—Y, where X is R¹—N(R²)—[R¹ and R² are the same or different and each is a hydrogen atom or an amino-protecting group J or R³—CH—N—[R³—CH is H—CH or a Shiff base type amino-protecting group]; and Y is —P(═O)(OH)—O—R⁴ [R⁴ is —CH₂—CH(O—R⁵)—CH2—O—R⁶ (R⁵ and R⁶ are the same or different and each is an acyl group having 2-30 carbon atoms or a hydrogen atom) or a hydrogen atom], a hydrogen atom or a hydroxy-protecting group.

In other embodiments, R¹ and R² are the same or different and each is a hydrogen atom, a halogen atom, a hydroxy group, an aryl group, an acyl group having 2˜30 carbon atoms, an alkyl group having 1-6 carbon atoms, an alkoxyl group having 1-6 carbon atoms, a hydroxyalkyl group having 1 -6 carbon atoms, a haloalkyl group having 1-6 carbon atoms, a haloalkoxyl group having 1-6 carbon atoms or a halohydroxyalkyl group having 1-6 carbon atoms, and R³ is a hydrogen atom, a halogen atom, a hydroxy group, an aryl group, an acyl group having 2-30 carbon atoms, an alkyl group having 1-6 carbon atoms, an alkoxyl group having 1-6 carbon atoms, a hydroxyalkyl group having 1-6 carbon atoms, a haloalkyl group having 1-6 carbon atoms, a haloalkoxyl group having 1-6 carbon atoms or a halohydroxyalkyl group having 1-6 carbon atoms.

Exemplary Etn analogs include phosphoethanolamine, monomethylethanolamine, dimethylethanolamine, N-acylphosphatidyl ethanolamine, phosphatidylethanolamine, and lysophosphatidylethanolamine and may include any of the Etn analogs.

As used herein, the term “Etn prodrug” refers to any compound that when administered to a biological system generates a biologically active Etn compound as a result of spontaneous chemical reaction(s), enzyme catalyzed chemical reaction(s), and/or metabolic chemical reaction(s), or a combination of each. Standard Etn prodrugs may be formed using groups attached to functionality, e.g. HO—, HS—, HOOC—, HOOPR₂—, associated with the drug, that cleave in vivo, Table 1 below represents various bonds that can be used to produce Etn pro-drugs or Etn hybrid molecules, as further discussed below.

TABLE 1
Chemical bonds that can be used to produce pro-drugs or hybrid molecules

| | |
|---|---|
| Bonds that are labile for hydrolysis Ethanolamlne can be linked through these bonds for producing a pro-drug and hybrid molecule | 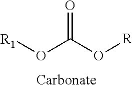 |
| | 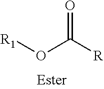 |
| | 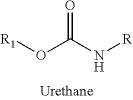 |
| | 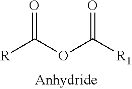 |

Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached Is an acyl group, an alkoxycarbonyl, aminocarbonyl, phosphate or sulfate, Etn prodrugs undergo a chemical transformation to produce the compound that is biologically active or is a precursor of the biologically active compound, In some cases, the prodrug is biologically active, usually less than the drug itself, and serves to improve drug efficacy or safety through improved oral bioavailability, pharmacodynamic half-life, etc. Exemplary Etn prodrugs are depicted in Table 2 below.

In certain embodiments further exemplified in Table 2 (i.e., molecule numbers x-y), the pharmaceutical composition includes a hybrid molecule of Etn and another chemotherapeutic drng. As used herein, the term “Etn hybrid” refers to For example, Etn hybrids of belinostat, panobinostat and vorinostat are shown in Table 2, molecule numbers 36 to 41, respectively, Any chemotherapeutic drug described herein may be used in a hybrid form with Etn provided that it contains a sufficient reactive group for forming the hybrid molecule with conjugation using an ester, carbonate, urethane, anhydride. The hydroxyl or amino group of Etn may be at the terminal end of the hybrid structure, Exemplary Etn hybrids include compounds listed in Table 2.

TABLE 2
Etn prodrugs and Etn hybrid molecules

| | |
|---|---|
| 1 | 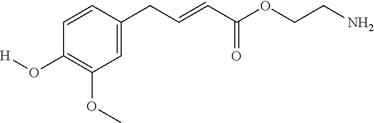 [SMILES: COc1cc(C/C=C/C(=O)OCCN)ccc1O] |
| | 2-aminoethyl (E)-4-(4-hydroxy-3-methoxy-phenyl)but-2-enoate |
| 2 | 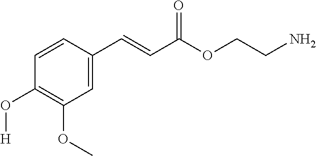 [SMILES: COc1cc(/C=C/C(=O)OCCN)ccc1O] |
| | 2-aminoethy (E)-3-(4-hydroxy-3-methoxy-phenyl)prop-2-enoate |
| 3 | 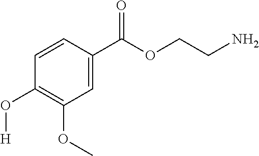 |
| | 2-aminoethyl 4-hydroxy-3-methoxy-benzoate |
| 4 | 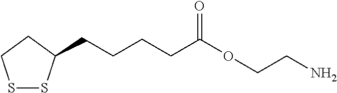 [SMILES: NCCOC(=O)CCCC[C@@H]1CCSS1] |
| | 2-aminoethyl 5-[(3R)-dithiolan-3-yl]pentanoate |
| 5 | 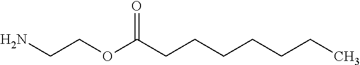 |
| | 2-aminoethyl octanoate |
| 6 | 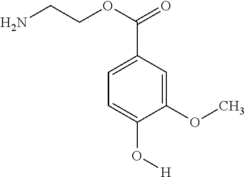 |
| | 2-aminoethyl 4-hydroxy-3-methoxy-benzoate |
| 7 | 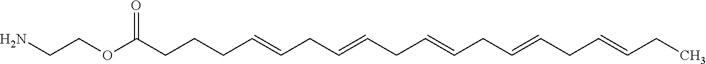 [SMILES: CC/C=C/C/C=C/C/C=C/C/C=C/C/C=C/CCCC(=O)OCCN] |
| | 2-aminoethyl (5E,8E,11E,14E,17E)-icosa-5,8,11,14,17-pentaenoate |
| 8 | 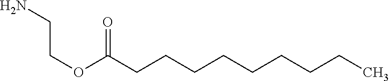 |
| | 2-aminoethyl decanoate |
| 9 | 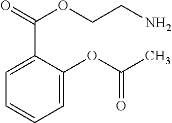 [SMILES: CC(=O)Oc1ccccc1C(=O)OCCN] |
| | 2-aminoethyl 2-acetoxybenzoate |
| 10 | 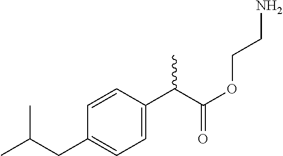 [SMILES: CC(C)Cc1ccc(C(C)C(=O)OCCN)cc1] |
| | 2-aminoethyl 2-(4-isobutylphenyl)propanoate |
| 11 | 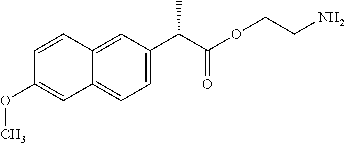 [SMILES: COc1ccc2cc([C@H](C)C(=O)OCCN)ccc2c1] |
| | 2-aminoethyl (2S)-2-(6-methoxy-2-naphthyl)propanoate |
| 12 | 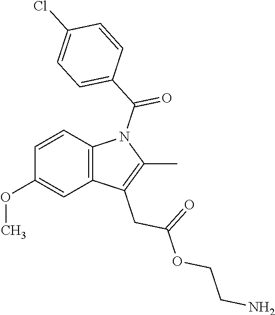 [SMILES: COc1ccc2c(c1)c(CC(=O)OCCN)c(C)n2C(=O)c1ccc(Cl)cc1] |
| | 2-aminoethyl 2-[1-(4-chlorobenzoyl)-5-methoxy-2-methyl-indol-3-yl]acetate |
| 13 | 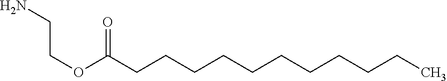 [SMILES: CCCCCCCCCCCC(=O)OCCN] |
| | 2-aminoethyl dodecanoate |
| 14 | 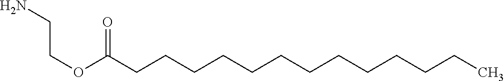 [SMILES: CCCCCCCCCCCCCC(=O)OCCN] |
| | 2-aminoethyl tetradecanoate |
| 15 | 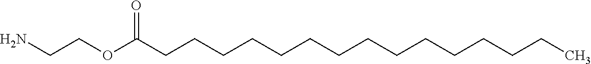 [SMILES: CCCCCCCCCCCCCCCC(=O)OCCN] |
| | 2-aminoethyl hexadecanoate |
| 16 | 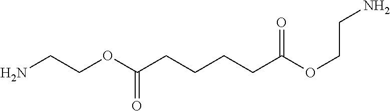 [SMILES: NCCOC(=O)CCCCC(=O)OCCN] |
| | bis(2-aminoethyl) hexanedioate |
| 17 | 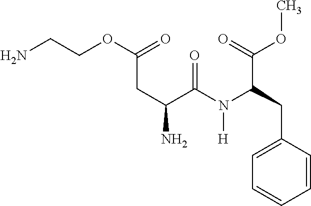 |
| | 2-aminoethyl (3S)-3-amino-4-[(1-benzyl-2-methoxy-2-oxo-ethyl)amino]-4-oxo- |
| | butanoate |
| 18 | 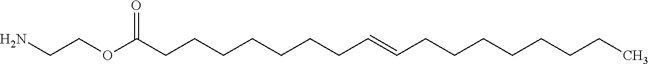 [SMILES: CCCCCCCC/C=C/CCCCCCCC(=O)OCCN] |
| | 2-aminoethyl (E)-octadec-9-enoate |
| 19 | 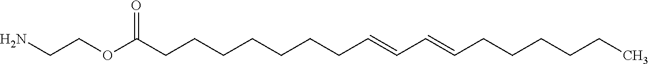 [SMILES: CCCCCC/C=C/C=C/CCCCCCCC(=O)OCCN] |
| | 2-aminoethyl (9E,11E)-octadeca-9,11-dienoate |
| 20 | 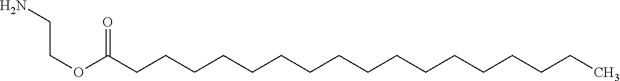 [SMILES: CCCCCCCCCCCCCCCCCC(=O)OCCN] |
| | 2-aminoethyl octadecenoate |
| 21 | 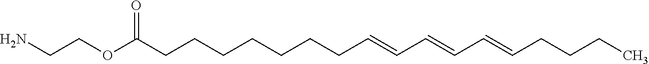 [SMILES: CCCC/C=C/C=C/C=C/CCCCCCCC(=O)OCCN] |
| | 2-aminoethyl (9E,11E,13E)-octadeca-9,11,13-trienoate |
| 22 | 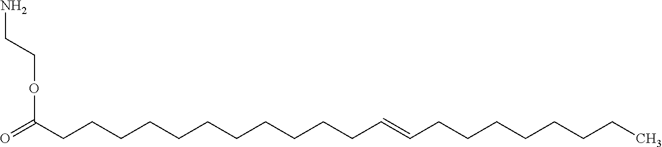 [SMILES: CCCCCCCC/C=C/CCCCCCCCCCCC(=O)OCCN] |
| | 2-aminoethyl (E)-docos-13-enoate |
| 23 | 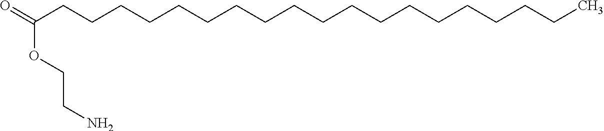 [SMILES: CCCCCCCCCCCCCCCCCCCC(=O)OCCN] |
| | 2-aminoethyl icosanoate |
| 24 | 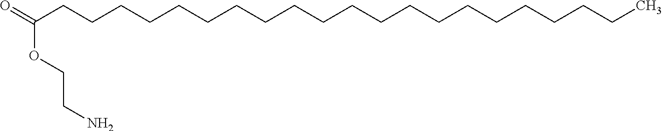 [SMILES: CCCCCCCCCCCCCCCCCCCCCC(=O)OCCN] |
| | 2-aminoethyl docosanoate |
| 25 | 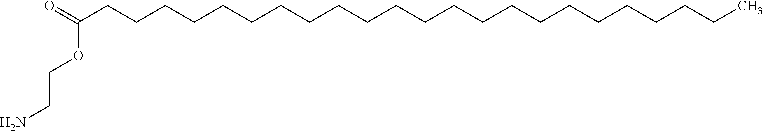 [SMILES: CCCCCCCCCCCCCCCCCCCCCCCC(=O)OCCN] |
| | 2-aminoethyl tetracosanoate |
| 26 | 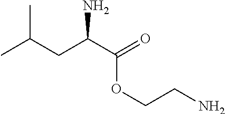 |
| | 2-aminoethyl (2R)-2-amino-4-methyl-pentanoate |
| 27 | 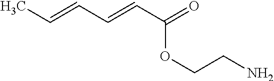 |
| | 2-aminoethyl (2E,4E)-hexa-2,4-dienoate |
| 28 | 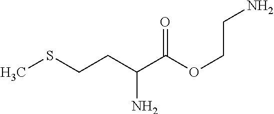 |
| | 2-aminoethyl 2-amino-4-methylsulfanyl-butanoate |
| 29 | 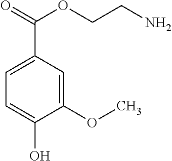 |
| | 2-aminoethyl 4-hydroxy-3-methoxy-benzoate |
| 30 | 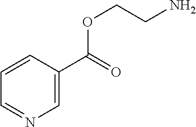 [SMILES: NCCOC(=O)c1cccnc1] |
| | 2-aminoethyl pyridine-3-carboxylate |
| 31 | 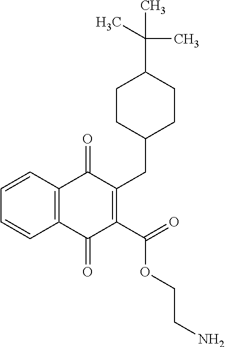 [SMILES: CC(C)(C)C1CCC(CC2=C(C(=O)OCCN)C(=O)c3ccccc3C2=O)CC1] |
| | 2-aminoethyl 3-[(4-tert-butylcyclohexyl)methyl]-1,4-dioxo-naphthalene-2- |
| | carboxylate |
| 32 | 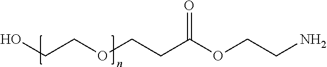 |
| | Pegylated ethanolamine |
| 33 | 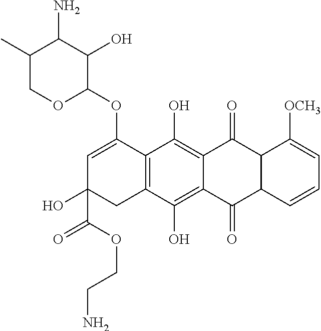 [SMILES: COC1=CC=CC2C(=O)c3c(O)c4c(c(O)c3C(=O)C12)C(OC1OCC(C)C(N)C1O)=CC(O)(C(=O)OCCN)C4] |
| | 2-aminoethyl 4-(4-amino-3-hydroxy-5-methyl-tetrahydropyran-2-yl)oxy-2,5,7,12- |
| | tetrahydroxy-6,11-dioxo-6a,10a- dihydro-1H-tetracene-2-carboxylate |
| 34 | 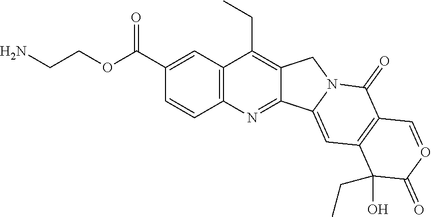 |
| 35 | 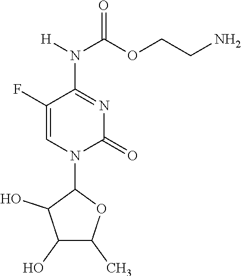 [SMILES: CC1OC(n2cc(F)c(NC(=O)OCCN)nc2=O)C(O)C1O] |
| | 2-aminoethyl N-[1-(3,4-dihydroxy-5-methyl-tetrahydrofuran-2-yl)-5-fluoro-2-oxo- |
| | pyrimidin-4-yl]carbamate |
| 36 | 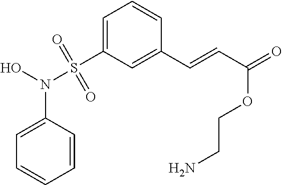 [SMILES: NCCOC(=O)/C=C/c1cccc(S(=O)(=O)N(O)c2ccccc2)c1] |
| | 2-aminoethyl (E)-3-[3-(phenylsulfamoyl)phenyl]prop-2-enoate |
| 37 | 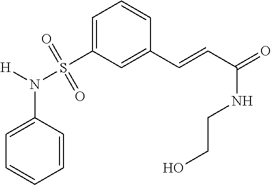 [SMILES: O=C(/C=C/c1cccc(S(=O)(=O)Nc2ccccc2)c1)NCCO] |
| | (E)-N-(2-hydroxyethyl)-3-[3-(phenylsulfamoyl)phenyl]prop-2-enamide |
| 38 | 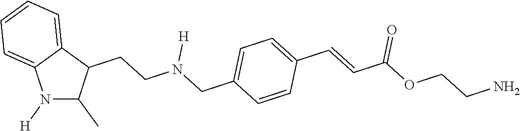 [SMILES: CC1Nc2ccccc2C1CCNCc1ccc(/C=C/C(=O)OCCN)cc1] |
| | 2-aminoethyl (E)-3-[4-[[2-(2-methylindolin-3-yl)ethylamino]methyl]phenyl]prop-2- |
| | enoate |
| 39 | 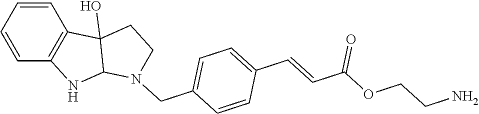 [SMILES: NCCOC(=O)/C=C/c1ccc(CN2CCC3(O)c4ccccc4NC23)cc1] |
| | 2-aminoethyl (E)-3-[4-[(8b-hydroxy-1,2,3a,4-tetrahydropyrrolo[2,3-b]indol-3- |
| | yl)methyl]phenyl]prop-2-enoate |
| 40 | 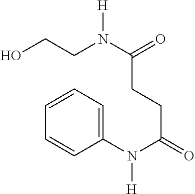 [SMILES: O=C(CCC(=O)Nc1ccccc1)NCCO] |
| | N-(2-hydroxyethyl)-N′-phenyl-butanediamide |
| 41 | 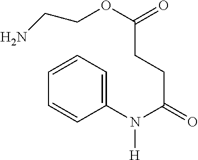 [SMILES: NCCOC(=O)CCC(=O)Nc1ccccc1] |
| | 2-aminoethyl 4-anilino-4-oxo-butanoate |
| 42 | 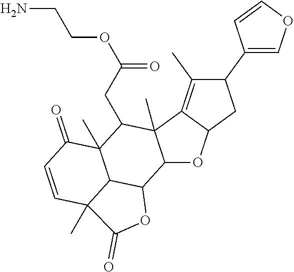 [SMILES: CC1=C2C(CC1c1ccoc1)OC1C3OC(=O)C4(C)C=CC(=O)C(C)(C34)C(CC(=O)OCCN)C21C] |
| 43 | 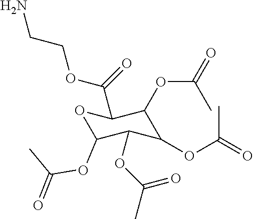 [SMILES: CC(=O)OC1OC(C(=O)OCCN)C(OC(C)=O)C(OC(C)=O)C1OC(C)=O] |
| | 2-aminoethyl 3,4,5,6-tetraacetoxytetrahydropyran-2-carboxylate |

In some embodiments, Etn is conjugated to a polymer. Examples of such polymers include, but are not limited to, polyethylene glycol (PEG), N-2-hydroxypropyl mehtacrylamide (HPMA), polyvinyl pyrrolidone (PVP), polyvinyl alcohol, polyglutamic acid (PGA), polymalic acid, glycylphenylalanylleucylglycine (GFLG)—lysosomal cleavage linker, dendrimers—polyethyleneimine and polyamido amine (PAMAM), polymeric micelles such as propylene oxide, L-lysine, caprolactone, D,L-lactic acid, styrene, aspartic acid, β-benzoyl-L-aspartate and spermine, biodegradable polymers such as poly (L-lysine), poly (L-glutamic acid) and poly (N-hydroxyalkyl)glutamine), carbohydrate polymers such as dextrins, hydroxyethyl starch (HES) and polysialic acid, smart polymers such as poly (acrylamide), poly (methylacrylic acid), poly (acrylic acid) and poly(2-(dimethylamino)ethyl methacrylate. Table 3 provides a classification of exemplary polymers for conjugation.

TABLE 3 Classification of exemplary polymers. Classification Polymer Natural Polymers Protein based polymers Collagen, albumin, gelatin Polysaccharides Agarose, alginate, carrageenan, hyaluronic acid, dextran, chitosan, cyclodextrins Synthetic polymers-Biodegradable Polyesters Poly(lactic acid), poly(glycolic acid), poly(hydroxyl butyrate), poly(ε-caprolactone), poly(β-malic acid), poly(dioxanones) Polyanhydrides Poly(sebacic acid), poly(adipic acid), poly(terphthalic acid) and various copolymers Polyamides Poly(imino carbonates), polyamino acids Phosphorous-based Polyphosphates, polyphosphonates, polymers polyphosphazenes Others Poly(cyano acrylates), polyurethanes, polyortho esters, polydihydropyrans, polyacetals Synthetic polymers—Non-biodegradable Cellulose derivatives Carboxymethyl cellulose, ethyl cellulose, cellulose acetate, cellulose acetate propionate, hydroxypropyl methyl cellulose Silicones Polydimethylsiloxane, colloidal silica Acrylic polymers Polymethacrylates, poly(methyl methacrylate), poly hydro (ethyl-methacrylate) Others Polyvinyl pyrrolidone, ethyl vinyl acetate, poloxamers, poloxamines

In some embodiments, the pharmaceutical composition comprises Etn or Etn conjugates in the form of nanosomes, liposome, noisome, nanoparticle, nanosphere, microsphere, microparticle, microemulsion, nanosuspension and/or micelles.

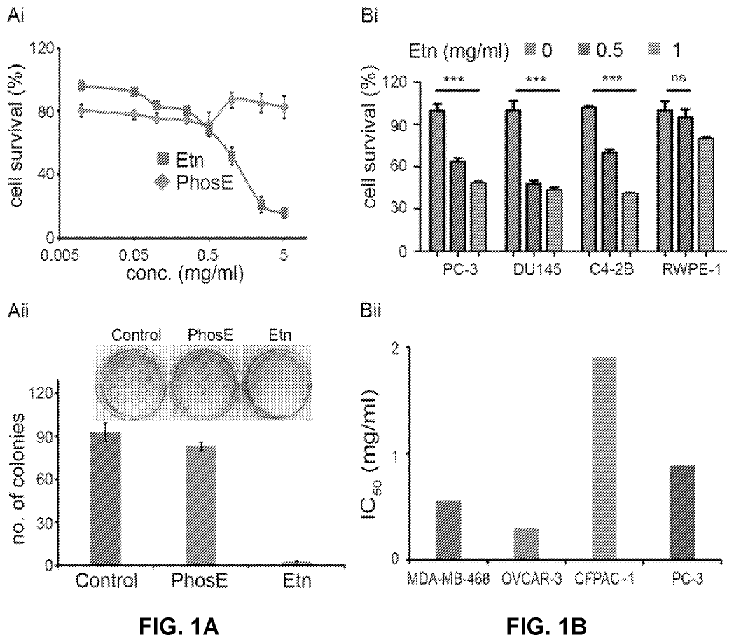

In other embodiments, the composition alternatively or additionally includes one or more substrate or product compounds of the Kennedy pathway of PE lipid biosynthesis (FIG. 1). Exemplary compounds include one or more members selected from the group consisting of PhosE, cytidine-diphosphoethanolamine (CDP-Etn), phosphatidylethanolamine, analogues therefrom, derivatives therefrom, and combinations thereof

In one embodiment, the composition further includes PhosE. In some embodiments, the composition includes PhosE in an amount that is 5% (w/w) or less, 10% (w/w) or less, 20% (w/w) or less, 30% (w/w) or less, 40% (w/w) or less, 50% (w/w) or less, 60% (w/w) or less, 70% (w/w) or less, 80% (w/w) or less, 90% (w/w) or less, or 100% (w/w) or less of the amount of Etn. In another embodiment, the composition is free of PhosE. As used herein, a composition is “free of PhosE” if the composition does not contain any PhosE, or contains PhosE at levels below 0.1% w/w.

In another embodiment, the composition alternatively or additionally includes one or more substrate or product compounds of the Kennedy pathway of phosphatidylserine, lipid biosynthesis. Exemplary compounds include one or more members selected from the group consisting of choline, phosphocholine, cytidine-diphosphocholine, phosphatidylcholine, analogous therefrom, derivatives therefrom, and combinations therefrom.

In certain embodiments, the patient is also administered one or more centrosome declustering agents, including but not limited to griseofulvin; noscapine, noscapine derivatives, such as brominated noscapine (e.g., 9-bromonoscapine), reduced bromonoscapine (RBN), N-(3-brormobenzyl) noscapine, aminonoscapine and water-soluble derivatives thereof; CW069; the phenanthridene-derived poly(ADP-ribose) polymerase inhibitor, PJ-34; N2-(3-pyridylmethyl)-5-nitro-2-furamide, N2-(2-thienylmethyl)-5-nitro-2-furamide, N2-benzyl-5-nitro-2-furamide, an anthracine compound as described in U.S. Patent Application Publication 2008/0051463; a 5-nitrofuran-2-carboxamide derivative as described in U.S. Provisional Application 61/619,780; and derivatives and analogs therefrom.

In others embodiments, the patient is also administered an inhibitor of HSET, a key mediator of centrosome clustering. In some embodiments, the inhibitor of HSET is a small molecule drug inhibiting the activity and/or expression of HSET in the targeted cell. Alternatively, or in addition, the patient may be administered an inhibitor of a protein that is upregulated with HSET or inhibitors of other proteins implicated in centrosome clustering. HSET co-regulated product targets include, but are not limited to Npap60L, CAS, Prc1, Ki67, survivin, phospho-survivin, Hif1α, aurora kinase B, p-Bcl2, Mad1, Plk1, FoxM1, KPNA2, Aurora A and combinations thereof. In other embodiments, the patient is administered one or more agents that block the nuclear accumulation of HSET during interphase.

In certain embodiments, the small molecule drug targets the motor domain of HSET and/or specifically binds to the HSET/microtubule binary complex so as to inhibit HSET's microtubule-stimulated and/or microtubule-independent ATPase activities. In a specific embodiment, the small molecule drug is AZ82 or CW069 or a therapeutically effective derivative, salt, enantiomer, or analog thereof.

AZ82 binds specifically to the KIFC1/microtubule (MT) binary complex and inhibits the MT-stimulated KIFC1 enzymatic activity in an ATP-competitive and MT-noncompetitive manner with a Ki of 0.043 μM. Treatment with AZ82 causes centrosome declustering in BT-549 breast cancer cells with amplified centrosomes.

Alternatively, or in addition, the patient may be administered with a poly(ADP-ribose) polymerase (PARP) inhibitor, an inhibitor of the Ras/MAPK pathway, an inhibitor of the P13K/AKT/mTOR pathway, an inhibitor of FoxM1, Hif1α, survivin, Aurora, Plk1 or a combination thereof. Exemplary PARP inhibitors include, but are not limited to olaparib, iniparib, velaparib, BMN-673, BSI-201, AG014699, ABT-888, GP121016, MK4827, INO-1001, CEP-9722, PJ-34, Tiq-A, Phen, PF-01367338 and combinations thereof. Exemplary Ras/MAPK pathway agents include, but are not limited to MAP/ERK kinase (MEK) inhibitors, such as trametinib, selumetinib, cobimetinib, CI-1040, PD0325901, AS703026, R04987655, R05068760, AZD6244, GSK1120212, TAK-733, U0126, MEK162, GDC-0973 and combinations thereof. Exemplary PI3K/AKT/mTOR pathway inhibitors include, but are not limited to everolimus, temsirolimus, GSK2126458, BEZ235, PIK90, P1103 and combinations thereof.

Anti-angiogenesis inhibitors include small molecule agents or antagonists targeting the VEGF pathway, the Tie2 pathway, or both. Exemplary small molecule antagonists of the VEGF pathway include multikinase inhibitors of VEGFR-2, including sunitinib, sorafenib, cediranib, pazonpanib and nintedanib. Tie2 binding antagonists also include the small molecule inhibitors, CGI-1842 (CGI Pharmaceuticals), LP-590 (Locus Pharmaceuticals), ACTB-1003 (Act Biotech/Bayer AG), CEP-11981 (Cephalon/Teva), MGCD265 (Methylgene), Regorafenib (Bayer), Cabozantinib/XL-184/BMS-907351 (Exelixis), Foretnib (Exelixis), MGCD-265 (MethylGene Inc.).

In recent years, a number of immune checkpoint regulators in the form of receptors and their ligands have been identified. Immune checkpoint regulators include, but are not limited to PD-1 and its ligands, PD-L1 and PD-L2; CTLA-4 and its ligands, B7-1 and B7-2; TIM-3 and its ligand, Galectin-9; LAG-3 and its ligands, including liver sinusoidal endothelial cell lectin (LSECtin) and Galectin-3; T cell Ig and ITIM domain (TIGIT) and its CD155 ligand; CD122 and its CD122R ligand; CD70, glucocorticoid-induced TNFR family-related protein (GITR), B7H3, B and T lymphocyte attenuator (BTLA), and VISTA (Le Mercier et al., Front. Immunol., (6), Article 418, 2015). In addition, a number of checkpoint regulator inhibitors have been identified and tested in various clinical and pre-clinical models and/or approved by the FDA (Kyi et al., FEBS Letters, 588:368-376 (2014). The concept of inhibitory receptor blockade, also known as immune checkpoint blockade, has been validated in humans with the approval of the anti-CTLA-4 antibody ipilimumab for metastatic melanoma.

Adjuvant chemotherapeutic compositions may also include wide variety of cytotoxic agents with different intracellular targets that can induce apoptosis. This means that the cytotoxic activity of cytotoxic drugs is not solely dependent on specific drug-target interaction, but also on the activity of apoptotic (cell signaling) machinery of the cancer cell. Examples of cytotoxic agents include, but are not limited to, platinum-based drugs (e.g., carboplatin, cisplatin, oxaliplatin, satraplatin, triplatin tetranin, and carboplatin etc.), natural phenols (e.g., cardamom, curcumin, galangal, ginger, melegueta pepper, turmeric, etc.), plant alkaloids and taxanes (e.g., camptothecin, docetaxel, paclitaxel, vinblastine, vincristine, virorelbine, vincristine, etc.), other alkylating agents (e.g., altretamine, busulfan, carmustine, chlorambucil, cyclophosphamide, dacarbazine, ethylenimines, haxmethyl melamine, hydrazines, ifosfamide, lomustine, mechlorethamine, melphalan, nitrosoureas, piperine, procarbazine, streptozocin, temozolomide, thiotepa, triazines, etc.), tumor antibiotics and anthracyclines (e.g., bleomycin, chromomycin, dactinomycin, daunorubicin, doxorubicin, epirubicin, idarubicin, mitomycin, mitoxantrone, plicamycin, etc.), topoisomerase inhibitors (e.g., amsacrine, etoposides, irinotecan, teniposides, toptecan, etc.), antimetabolites (e.g., 5-fluorouracil, 6-thioguanine, 6-mercaptopurine, adenosine deaminase inhibtors, capecitabine, cladribine, cytarabine, foxuridine, fludarabine, gemcitabine, methotrexate, nelerabine, pentaostatin mitotic inhibitor, purine antagonists, pyrimidine antagonists, etc.), miscellaneous anticancer agents (e.g., ixabepilone, asparaginase, bexarotene, estramustine, hydroxyurea, isotretinoin, mitotane, pegaspargase, retinoids, tretinoin, etc.), combinations thereof, and pharmaceutically acceptable salts thereof.

Because of its basic amino group and the hydroxyl group, Etn has properties resembling those of both amines and alcohols. Thus, they can form salts with acids, and the hydroxyl group permits ester formation. When Etn reacts with organic acids, salt formation always takes place in preference to ester formation.

In certain embodiments, the active agent(s), including Etn, may be administered as a pharmaceutically acceptable salt. The active agents may be administered as an inorganic acid salt, organic acid salt or an organic-substituted inorganic acid salt. As used herein, the term “pharmaceutically acceptable salt” means a salt prepared from a base or an acid which is acceptable for administration to a patient, such as a mammal (for example, salts having acceptable mammalian safety for a given dosage regime). Pharmaceutically acceptable salts can be derived from pharmaceutically acceptable inorganic or organic acids or from pharmaceutically acceptable inorganic or organic bases.

Pharmaceutically acceptable acid addition salts may be prepared from inorganic acids, organic acids or organic-substituted inorganic acids. Salts derived from pharmaceutically acceptable inorganic acids include salts of boric acid, carbonic acid, hydrohalic acids (e.g., hydrobromic acid, hydrochloric acid, hydrofluoric acid or hydroiodic acid); nitric acid, phosphoric acid, sulfamic acid, sulfuric acid, and the like.

Salts derived from pharmaceutically acceptable organic acids include salts of aliphatic hydroxyl acids (for example, citric acid, gluconic acid, glycolic acid, lactic acid, lactobionic acid, malic acid, and tartaric acid); aliphatic monocarboxylic acids (for example, acetic acid, butyric acid, formic acid, propionic acid and trifluoroacetic acid); amino acids (for example, aspartic acid and glutamic acid); aromatic carboxylic acids (for example, benzoic, p-chlorobenzoic acid, diphenylacetic acid, gentisic acid, hippuric acid, and triphenylacetic acid), aromatic hydroxyl acids (for example, o-hydroxybenzoic acid, p-hydroxybenzoic acid, 1-hydroxynaphthalene-2-carboxylic acid and 3-hydroxynaphthalene-2-carboxylic acid); ascorbic acid, dicarboxylic acids (for example, fumaric acid, maleic acid, oxalic acid and succinic acid); glucuronic acid, mandelic acid, mucic acid, nicotinic acid, orotic acid, pamoic acid, pantothenic acid; sulfonic acids (for example, benzenesulfonic acid, camphosulfonic acid, edisylic acid, ethanesulfonic acid, isethionic acid, methanesulfonic acid, naphthalenesulfonic acid, naphthalene-1,5-disulfonic acid, naphthalene-2,6-disulfonic acid and p-toluenesulfonic acid); xinafoic acid, and the like.

Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The compositions may be further distinguished by their pH. In some embodiments, the composition is in a liquid form with a pH between 2.0-8.0, between 3.0-7.0, between 4.0-6.0, between 4.0-5.0, between 4.5-5.5, between 5.0-6.0, between 5.5-6.5, between 6.0-7.0, between 6.5-7.5, between 7.0-8.0, between 7.5-8.5, between 8.0-9.0, or between any range defined by any of these pH values. In some embodiments, the composition has a pH of about 4, 5, 6, 7, 8 or 9. In some embodiments, the composition has a pH of about 5. In some embodiments, the composition has pH of about 7.4.

As used herein, the “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In a preferred embodiment, the composition is orally administered. Methods for making formulations for oral administration are found, for example, in “Remington: The Science and Practice of Pharmacy” (20th ed., ed. A. R. Gennaro, 2000, Lippincott Williams & Wilkins). Oral compositions generally include an edible carrier, an inert diluent, or both. Formulations for oral administration include e.g., tablets, pills, caplets, hard capsules, soft capsules, sachets, and liquid dosage forms, and may contain various additives and/or excipients as needed. In addition, liquid-filled capsules can include the active agent(s) of the present disclosure.

When administered in solid form, the composition may include a solid carrier. The carrier may comprise a porous excipient and optionally a binder and/or disintegrant. When the solid carrier is in the form of granules, the median particle size of the granules may range from about 5 microns to about 600 microns, for example from about 10 to about 300 microns. Granules may be compressed to form a tablet which is used as the solid carrier.

The porous excipient typically forms the bulk of the solid carrier. The porous excipient (and the solid carrier) has a porosity of, for example, greater than about 10% v/v, such as greater than about 15% v/v, greater than about 20% v/v, greater than about 30% v/v or greater than about 30% v/v. In a preferred embodiment, the porosity is greater than about 30% v/v, for example, from about 30 to about 50% v/v. In another embodiment, the porosity is up to about 97% (e.g., from about 90 to about 94%) (such as Zeopharm or Aeroperl).

The porous excipient may have a median particle size of from about 5 microns to about 600 microns, for example from about 10 to about 300 microns. In one embodiment, the porous excipient may have a particle size of from about 10 microns to about 150 microns.

The solid carrier may include the porous excipient at a concentration of about 20% w/w or more, such as about 25% w/w or more, about 30% w/w or more, about 35% w/w or more, about 40% w/w or more, about 45% w/w or more, about 50 w/w or more, about 60% w/w or more, about 70% or more, about 80% or more, about 90% or more, about 95% or more, 98% or more, or any range of percentages there between.

Exemplary porous excipients include, but are not limited to, metal oxides, metal silicates, metal carbonates, metal phosphates, metal sulfates, sugar alcohols, sugars, celluloses, cellulose derivatives, and any combination of those. In a preferred embodiment, the porous excipient is a metal silicate, e.g., a silicon dioxide, such as Zeopharm (available from J. M. Huber Corporation) or Aeroperl (available from Evonik industries). In another preferred embodiment, the porous excipient is a metal oxide, such as magnesium aluminometasilicate.

Metal oxides include as examples, but are not limited to, magnesium oxide, calcium oxide, zinc oxide, aluminum oxide, titanium dioxide (such as Tronox A-HP-328 and Tronox A-HP-100), silicon dioxides (such as Aerosil, Cab-O-Sil, Syloid, Aeroperl, Sunsil (silicon beads), Zeofree, Zeopharm, Sipernat), and mixtures thereof. In one embodiment, the metal oxide is titanium dioxide, silicon dioxide or a mixture thereof. Silicon dioxides may be subdivided into porous and nonporous silicas.

Metal silicates include as examples, but are not limited to, sodium silicate, potassium silicate, magnesium silicate, calcium silicate including synthetic calcium silicate such as, e.g., Hubersorp, zinc silicate, aluminum silicate, sodium aluminosilicate such as, e.g., Zeolex, magnesium aluminum silicate, magnesium aluminum metasilicate, aluminium metasilicate. The porous excipient may be a hydrous aluminum silicate or alkaline earth metal silicate, such as magnesium aluminum metasilicate (e.g., Neusilin available from Fuji Chemical Co.).

Suitable metal phosphates include, but are not limited to, sodium phosphate, disodium hydrogen phosphate, sodium dihydrogen phosphate, potassium phosphate, dipotassium hydrogen phosphate, potassium dihydrogen phosphate, calcium phosphate, magnesium phosphate, zinc phosphate, aluminum phosphate, and combinations thereof. For example, the porous excipient can be dibasic anhydrous calcium phosphate, dibasic dihydrate calcium phosphate, tribasic calcium phosphate, or a combination thereof.

Exemplary metal sulfates include, e.g, sodium sulfate, sodium hydrogen sulfate, potassium sulfate, potassium hydrogen sulfate, calcium sulfate, magnesium sulfate, zinc sulfate aluminum sulfate, and mixtures thereof.

Exemplary sugar alcohols include, e.g., sorbitol, xylitol, mannitol, maltitol, inositol, and/or it may be a sugar selected from the group consisting of mono-, di- or polysaccharides including saccharose, glucose, fructose, sorbose, xylose, lactose, dextran, dextran derivatives, cyclodextrins, and mixtures thereof.

Exemplary celluloses and cellulose derivatives include, e.g., cellulose, microcrystalline cellulose, cellulose derivatives including porous cellulose beads: cellulose, hydroxypropyl methylcellulose (HPMC), hydroxypropyl cellulose (H PC), methylcellulose, ethylcellulose, sodium carboxymethylcellulose, hydroxyethyl cellulose etc.

The solid oral dosage form may further comprise one or more pharmaceutically acceptable excipients. Examples of such excipients include, but are not limited to, fillers, diluents, binders, lubricants, glidants, enhancers, wetting agents, surfactants, antioxidants, metal scavengers, pH-adjusting agents, acidifying agents, alkalizing agents, preservatives, buffering agents, chelating agents, stabilizing agents, coloring agents, complexing agents, emulsifying and/or solubilizing agents, absorption enhancing agents, modify release agents, flavoring agents, taste-masking agents, humectants, and sweetening agents.

The amount of solid carrier in the solid oral dosage form may vary depending on its porosity, as the liquid formulation. Since the solid oral dosage form, such as tablet or capsule, is intended for oral ingestion by a mammal, such as a human subject, the solid oral dosage form preferably weighs from about 500 mg to about 5000 mg, such as from about 600 mg to about 2000 mg, or from about 600 mg to about 1500 mg. In one embodiment, the solid oral dosage form weighs from about 700 mg to about 1200 mg.

The solid oral dosage form (e.g., oral tablet) described herein may optionally contain one or more coatings, such as a sub-coating and/or modified release coating (e.g. an enteric coating). The sub-coating may be, e.g., Opadray AMB OY-B. The enteric coating may contain, e.g., Acryl EZE, dimethicone and triethyl citrate.

In one embodiment, the solid oral dosage form does not have a coating. In a preferred embodiment, the solid oral dosage form does not have an enteric coating. In another embodiment, the solid oral dosage form does not have a modified release coating. In certain embodiments, the solid oral dosage form provides for immediate release of the active agent(s). In other embodimens, the solid oral dosage form provides extended release of the active agent(s).

The solid oral dosage form may be in the form of a tablet. In one embodiment, the tablet is a compressed or molded tablet, e.g., having a hardness of from about 20 N to about 150 N. The hardness of the tablet can be from about 30, 40, or 50 N to about 70, 80, 90 or 100 N.

The oral tablet may include one or more excipients, such as those mentioned above including, but not limited to, flavoring agents, lubricants, binders, preservatives, and disintegrants.

In some embodiments, the active agents are adsorbed onto a nanoparticle or solid matrix (e.g., a porous silicate including alkali-metal silicates, alkaline earth metal silicates, or aluminum silicates, or including aluminum silicate, magnesium aluminum silicate, sodium silicate, potassium silicate, magnesium silicate, or calcium silicate), or any other solid matrix described herein. In certain embodiments, the active agent(s) are incorporated into or onto a nanoparticle. As used herein, the term “nanoparticle” refers to a solid particle having a structure including at least one region or characteristic dimension with a dimension of between 1-500 nm and having any suitable shape, e.g., a rectangle, a circle, a sphere, a cube, an ellipse, or other regular or irregular shape. Non-limiting examples of suitable nanoparticles may include liposomes, poloxamers, microemulsions, micelles, dendrimers and other phospholipid-containing systems, and perfluorocarbon nanoparticles. The term “nanoparticle” can include nanospheres, nanorods, nanoshells, and nanoprisms and these nanoparticles can be part of a nanonetwork. Without limitations, the nanoparticles used herein can be any nanoparticle available in the art or available to one of skill in the art.

In some embodiments, the nanoparticle is of size from about 10 nm to about 750 nm, from about 20 nm to about 500 nm, from about 25 nm to about 250 nm, or from about 50 nm to about 150 run. In some embodiments, the nanoparticle is of size from about 5 nm to about 75 nm, from about 10 nm to about 50 nm, from about 15 nm to about 25 nm. The nanoparticles can be, e.g., monodisperse or polydisperse and the variation in diameter of the particles of a given dispersion can vary. The nanoparticles can be hollow or solid. In some embodiments, the nanoparticles have an average diameter of less than 500 run, less than 300 nm, less than 100 nm, less than 50 nm, less than 25 nm, less than 10 nm or less than 5 nm.

Nanoparticles can be made, for example, out of metals such as iron, nickel, aluminum, gold, copper, zinc, cadmium, titanium, zirconium, tin, lead, chromium, manganese and cobalt; metal oxides and hydrated oxides such as aluminum oxide, chromium oxide, iron oxide, zinc oxide, and cobalt oxide; metal silicates such as of magnesium, aluminum, zinc, lead, chromium, copper, iron, cobalt, and nickel; alloys such as bronze, brass, stainless steel, and so forth. Nanoparticles can also be made of non-metal or organic materials such as cellulose, ceramics, glass, nylon, polystyrene, rubber, plastic, or latex. In some embodiments, nanoparticles comprise a combination of a metal and a non-metal or organic compound, for example, methacrylate- or styrene-coated metals and silicate coated metals. The base material can be doped with an agent to alter its physical or chemical properties. For example, rare earth oxides can be included in aluminosilicate glasses to create a paramagnetic glass materials with high density (see White & Day, Key Engineering Materials Vol. 94-95, 181-208, 1994). In some embodiments, nanoparticles comprise or consist of biodegradable organic materials, such as cellulose, dextran, and the like. Suitable commercially available particles include, for example, nickel particles (Type 123, VM 63, 18/209A, 10/585A, 347355 and HDNP sold by Novamet Specialty Products, Inc., Wyckoff, N.J.; 08841R sold by Spex, Inc.; 01509BW sold by Aldrich), stainless steel particles (P316L sold by Ametek), zinc dust (Aldrich), palladium particles (D13A17, John Matthey Elec.), and TiO₂, SiO₂, or MnO₂ particles (Aldrich).

In some embodiments, the nanoparticles are freeze-dried to form solid dried nanoparticles. The dried nanoparticles may be loaded in a capsule (such as a two-part hard gelatin capsule) for oral administration in a subject. In addition, the capsule may be further coated with an enteric coating. The freeze-dried nanoparticles can be rehydrated in solution or by contacting fluid so to revert to wet nanoparticles having positive surface charge.

In some embodiments, a liposome delivery vehicle may be utilized. Liposomes, depending upon the embodiment, are suitable for delivery of the active agents in the present disclosure in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tetradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyI)-N,N,N-trimethyl ammonium chloride, 1,1′-dioctadecyl-3,3,3′,3′-tetramethylindocarbocyanine, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which sphingosine is the structural counterpart of glycerol and one of the fatty acids of a phosphoglyceride, or cholesterol, a major component of animal cell membranes. Liposomes may optionally contain pegylated lipids, which are lipids covalently linked to polyethylene glycol (PEG) or derivatives thereof. Exemplary PEGs can have a molecular weight of 200-10,000 kDa (e.g., 400-4000 kDa, 500-1000 kDa, 750-1500 kDa, 800-1200 kDa, 900-1100 kDa, or about 1000 kDa). PEG derivatives include, for example, methylated PEG, polypropylene glycol (PPG), PEG-NHS, PEG-aldehyde, PEG-SH, PEG-NH₂, PEG-CO₂H, PEG-OMe and other ethers, branched PEGs, and PEG copolymers (e.g., PEG-b-PPG-b-PEG-1100, PEG-PPG-PEG-1900, PPG-PEG-MBE-1700, and PPG-PEG-PPG-2000).

Liposomes may further comprise a suitable solvent. The solvent may be an organic solvent or an inorganic solvent. Suitable solvents include, but are not limited to, di methylsulfoxide (DMSO), methylpyrrolidone, N-methylpyrrolidone, acetronitrile, alcohols, dimethylformamide, tetrahydrofuran, or combinations thereof

Liposomes may be prepared by any known method of preparing liposomes for drug delivery, such as, for example, detailed in e.g., U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618. For example, liposomes may be prepared by sonicating lipids in an aqueous solution, solvent injection, lipid hydration, reverse evaporation, or freeze drying by repeated freezing and thawing. In certain preferred embodiments the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, the composition is delivered to a tissue or cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear, but also may appear as a milky colloidal suspension depending on exact composition, storage conditions, pH, temperature, surface charge, shape, and such. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions may optimally comprise phospholipids, although other hydrophobic core components singularly or in mixtures (e.g., perfluorocarbons: see below) may contribute to the composition of the particle. Any of the phospholipids detailed above for liposomes are suitable for embodiments directed to microemulsions. The composition of the invention may be encapsulated in a microemulsion by any method generally known in the art.

In yet another embodiment, the composition may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate or conjugate the active agents of the present disclosure via standard linker chemistries known in the art. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

In certain embodiments, the nanoparticle is a perfluorocarbon nanoparticle. Such nanoparticles are known in the art. For instance, see e.g., U.S. Pat. Nos. 5,690,907; 5,780,010; 5,989,520 and 5,958,371. Exemplary perfluorocarbon emulsions are disclosed in e.g., U.S. Pat. Nos. 4,927,623; 5,077,036; 5,114,703; 5,171,755; 5,304,325; 5,350,571; 5,393,524 and 5,403,575 and include those in which the perfluorocarbon compound is perfluorodecalin, perfluorooctane, perfluorodichlorooctane, perfluoro-n-octyl bromide, perfluoroheptane, perfluorodecane, perfluorocyclohexane, perfluoromorpholine, perfluorotripropylamine, perfluortributylamine, perfluorodimethylcyclohexane, perfluorotrimethylcyclohexane, perfluorodicyclohexyl ether, perfluoro-n-butyltetrahydrofuran, and compounds that are structurally similar to these compounds and are partially or fully halogenated (including at least some fluorine substituents) or partially or fully perfluorinated including perfluoroalkylated ether, polyether or crown ether. In some embodiments, the perfluorocarbon compound is perfluoro-n-octyl bromide. In other embodiments, the perfluorocarbon compound may be a perfluoroalkylated crown ether.

In some embodiments, the nanoparticle comprises on its surface a biocompatible layer or material. As used herein, the term “biocompatible layer or material” refers to any material or layer that does not deteriorate appreciably and does not induce a significant adverse effect, e.g., toxic reaction, over time when placed adjacent to the biological tissue of a subject, or induce blood clotting or coagulation when it comes in contact with blood. Suitable biocompatible materials can include, but are not limited to, polymers comprising an amino group (e.g., carbohydrate-based amino-polymers, protein-based amino-polymers, or molecules comprising at least one amino group), silk fibroin, derivatives and copolymers of polyimides, polyvinyl alcohol, polyethyleneimine, polyvinylamine, polyacrylates, polyamides, polyesters, polycarbonates, polydimethylsiloxane, polyimide, polyethylene terephthalate, polymethylmethacrylate, polyurethane, polyvinylchloride, polystyrene, polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ketones), poly(ethylene glycol), styrene-acrylonitrile resin, poly(trimethylene terephthalate), polyvinyl butyral, polyvinylidenedifluoride, poly(vinyl pyrrolidone), polyethylene glycol, natural or synthetic phospholipids, fatty acids, cholesterols, lysolipids, sphingomyelins, and the like, including lipid conjugated polyethylene glycol. Various commercial anionic, cationic, and nonionic surfactants can also be employed, including Tweens, Spans, Tritons, and the like. Some surfactants are themselves fluorinated, such as perfluorinated alkanoic acids such as perfluorohexanoic and perfluorooctanoic acids, perfluorinated alkyl sulfonamide, alkylene quaternary ammonium salts and the like. In addition, perfluorinated alcohol phosphate esters can be employed. Cationic lipids, including DOTMA, N-[1-(2,3-dioleoyloxy)propyl]-N,N,N-trimethylammonium chloride; DOTAP, 1,2-dioleoyloxy-3-(trimethylammonio)propane; DOTB, 1,2-dioleoyl-3-(4′-trimethyl-ammonio)butanoyl-sn-glycero1,2-diacyl-3-tr-imethylammonium-propane; 1,2-diacyl-3-dimethylammonium-propane; 1,2-diacyl-sn-glycerol-3-ethyl phosphocholine; and 3.beta.-[N′,N′-dimethylaminoethane)-carbamol]cholesterol-HCI, may also be usedand any combinations thereof.

In certain preferred embodiments, a nanoparticle can comprise on its surface a biocompatible layer to prolong the circulation time of the nanoparticles in a subject, such as polyethylene gycol (PEG). In some embodiments, the biocompatible layer can be selected to induce antigen-specific immunity in a subject. In other embodiments, the biocompatible layer can be selected to reduce or minimize the exposure of the nanoparticle material to surrounding tissue in a subject.

Exemplary nanoparticle compositions for use in the present methods are described in U.S. Patent Publication Application Nos. 2007/0154559, 2010/0104645 and 2015/0150822.

The pharmaceutical compositions of the present disclosure may further include one or more absorption enhancers to enhance the efficiency of transport through the intestinal mucosa into the blood. In one embodiment, the absorption enhancer includes an oil coating that constitutes a physical barrier providing additional protection against digestive enzymes. Secretion of bile acids typically causes dispersion of the oil suspension into smaller particles, which can be absorbed in the small intestine. While the particle size is reduced after traversing the stomach and entering the small intestine, the particles remain in a size range of 30-1000 nm, too large to be a substrate for lipases and peptidases, preserving the protective effect of the composition. Advantageously, lipid-coating particles of this size are absorbed to chylomicrons by lacteal vessels, which are lymphatic vessels originating in the villi of the small intestine. Particles absorbed in this manner can reach the bloodstream without undergoing first-pass metabolism.

In other embodiments, the absorption enhancer(s) include one or more bile salts, anionic surfactants, medium-chain fatty acids, phosphate esters and sodium N-[8-(2-hydroxybenzoyl)amino]caprylate.

In other embodiments, oral availability of the active agent(s) may be enhanced by including an include an acyl carnitine (e.g., palmitoyl carnitine), optionally in combination with an alcohol, a polysorbate surfactant, a carboxylic acid, an alcohol, a polyethylene glycol, a polyglycolized glyceride, alkyl saccharides, ester saccharides, a TPGS compound, or a sugar, as described in U.S. Patent Publication Application No. 2016/0074322.

In some embodiments, the composition may be further coated, conjugated to or modified with a tumor-specific or cell/tissue specific targeting agent for selective targeting of cancer cells. The targeting agent may be a small molecule (e.g., folate, adenosine, purine, lysine), peptide, ligand, antibody fragment, aptamer or synbody. Such compositions may allow for the use of a lower dose of cytotoxic drugs, reduce adverse events, increase efficacy, and reduce the possibility of the drugs being rapidly cleared from targeted tumors or cancer cells. Targeted compositions according to the present application allow for active agents to be taken up by cancer cells so as to effectively deliver the active agents to intracellular targets in the cancer cells to promote apoptosis and limit the potential of chemoresistance and systemic toxicities.

In some embodiments, the cell targeting agent is directed to tumor associated antigen, preferably a cell surface antigen. Examples of tumor associated antigens include, but are not limited to, adenosine receptors, alpha v beta 3, aminopeptidase P, alpha-fetoprotein, cancer antigen 125, carcinoembryonic antigen, cCaveolin-1, chemokine receptors, clusterin, oncofetal antigens, CD20, epithelial tumor antigen, melanoma associated antigen, Ras, p53, Her2/Neu, ErbB2, ErbB3, ErbB4, folate receptor, prostate-specific membrane antigen, prostate specific antigen, purine receptors, radiation-induced cell surface receptor, serpin B3, serpin B4, squamous cell carcinoma antigens, thrombospondin, tumor antigen 4, tumor-associated glycoprotein 72, tyosinase, and tyrosine kinases. In certain preferred embodiments, the cell targeting agent is folate or a folate derivative that binds specifically to folate receptors (FRs).

The reduced folate carrier (RFC) system is a low-affinity, high capacity system that mediates the uptake of reduced folates into cancer cells at pharmacologic (pM) concentrations. The concentration of physiologic folates is in the range of 5 to 50 nM. Therefore, high affinity human FRs exist and are encoded by a family of genes whose homologous products are termed FR type α, β, γ, or δ, which are also described as FR1, FR2, FR3, or FR4, respectively. The membrane isoforms FR1, FR2, and FR4 can bind and transport folate or folate derivatives into the cell, while FR3 lacks a membrane anchor and is secreted from the cell. FR1 and FR2 bind folate and 6S 5-formyltetrahydrofolate (i.e., leucovorin) with similar yet different affinities 1.5 nM versus 0.35 nM (folate) and 800 nM versus 7 nM (leucovorin), respectively. 6S 5-methyltetrahydrofolate is the predominate folate in the blood and has similar affinities for FR1 and FR2, 55 nM and 1 nM, respectively.

In certain compositions, especially those for non-oral delivery, the targeting agent may be an antibody or peptide capable of binding tumor associated antigens.

In certain embodiments, the pharmaceutical composition is orally administered as non-toxic anticancer formulation comprising monoethanolamine (Etn), an Etn prodrug, an Etn hybrid molecule, or a combination thereof. In some embodiments, the pharmaceutical composition is orally administered as non-toxic anticancer formulation comprising monoethanolamine (Etn) and phosphoethanolamine (PhosE).

As used herein, the term “pharmaceutically acceptable carrier” include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. See e.g., A.H. Kibbe Handbook of Pharmaceutical Excipients, 3rd ed. Pharmaceutical Press, London, UK (2000). Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin. In some embodiments, the pharmaceutical composition of the present application comprises Etn, a phosphate salt, salts, and a pharmaceutically acceptable carrier.

The pharmaceutical composition is formulated to be compatible with its intended route of administration. The compounds may be administered to the patient with known methods, such as oral administration, intravenous administration as a bolus or by continuous infusion over a period of time, by intramuscular, intraperitoneal, intracerebrospinal, subcutaneous, intra-articular, intrasynovial, intrathecal, topical, transmucosal and/or inhalation routes. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

In certain embodiments, compositions for oral delivery may include one or more structural elements promoting adherence to the intestinal mucosa after oral administration, thereby significantly increasing the time of intestinal transit of the formulation. In some embodiments, the composition is formulated as a solid or semi-solid formulation in capsules.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Therapeutic efficacy and toxicity of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. In certain embodiments, single dosage contains 0.01 ug to 50 mg of the active compound.

As a general proposition, the therapeutically effective amount of the active compound will be in the range of about 1 ng/kg body weight/day to about 100 mg/kg body weight/day whether by one or more administrations. In a particular embodiments, the active compound is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 pg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 pg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 pg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In certain embodiments, the active compound is administered at a dose of 500 μg to 20 g every three days, or 10 μg to 400 mg/kg body weight every three days. In other embodiments, the active compound is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 pg to about 1000 mg per injection, about 1 μg to about 10,000 mg per individual administration, about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μg to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The therapeutic agent(s) may be administered daily, or every 2, 3, 4, 5, 6 or 7 days, or every 1, 2, 3 or 4 weeks.

In other particular embodiments, the active compound is administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage(s) will be dependent on the condition, size, age and condition of the patient.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Ethanolamine Gormulation for Treating Ovarian Serous and Clear Cell Carcinoma

Epithelial ovarian cancer (EOC) is a life-threatening disease characterized by late-stage presentation; EOCs are therefore a leading cause of death for gynecological cancers. The standard treatment for EOCs is debulking surgery followed by platinum-based chemotherapy. While these treatments are often initially efficacious, most patients develop recurrent disease, a largely incurable state. Ovarian clear cell carcinomas (OCCCs), a subtype of EOCs, are characterized by clear cells with aberrant lipid and glycogen accumulation. OCCC comprises 5-10% of ovarian carcinomas in North America, and -25% of EOCs in Japan. It frequently presents in perimenopausal women, and is often associated with endometriosis, thromboembolic vascular complications, and hypercalcemia. In contrast to high grade serous ovarian carcinoma, OCCC is usually detected in an early stage (stage I). Nonetheless, advanced stage/recurrent patients with OCCC have a much poorer prognosis than patients with other EOC subtypes mainly because the former are refractory to platinum-based regimens. Hence, there is an urgent unmet need for new OCCC treatment paradigms.

Chemoresistance stems from the tumor's ability to reprogram cellular metabolism to overcome metabolic stress imposed by the tumor microenvironment (TME). As for many other cancer types, OCCC cells become dependent on these metabolic changes, which could potentially be exploited to identify novel therapeutic targets. Monotherapy with immune checkpoint inhibitors (las) has so far yielded disappointing results in ovarian cancer when compared to other solid tumors. To improve response, multiple trials are underway combining las with drugs affecting other targets. Two immunotherapy studies from 2015 demonstrated responses in the small numbers of OCCC patients enrolled. OCCC and renal cell carcinomas (RCCs) share similar gene expression profiles and currently, Nivolumab, an ICI, is FDA-approved for RCC; thus, Nivolumab may merit further exploration in OCCC. One factor contributing to the ineffectiveness of immunotherapies in ovarian cancers could be TME hypoxia, which changes the antigen-presenting properties of myeloid cells, increases PD-L1 expression in myeloid-derived suppressor cells, induces suppression of T effector cells, and promotes generation and maintenance of Tregs. OCCCs express high levels of hypoxia-inducible factor-lalpha (HIF-Iα), which activates genes that promote angiogenesis, resistance to anti-tumor therapy, and cell survival. The simple lipid monoethanolamine (Etn) exhibits robust in vitro and in vivo efficacy in prostate cancer cell lines and xenograft models, respectively, and in breast, colon, pancreatic and ovarian cancer cell lines, while remaining non-toxic to healthy cells. Essentially, Etn acts as a pro-drug, which enters tumor cells and is converted into the cytotoxic lipid phosphoethanolamine (PhosE). This ATP-dependent conversion of Etn into PhosE is primarily catalyzed by the enzyme choline kinase (CK), which is overexpressed in multiple cancer types including prostate and ovarian cancers. Importantly, Etn treatment triggers a stark downregulation of HIF-1α, glucose, glutamine, and oxygen consumption rate (OCR) in tumor cells, alters lipid biosynthesis/accumulation and membrane compositions/morphology, and precipitates a catastrophic uncoupling of multiple pathways to induce metabolic crisis and cell death. The ovarian cancer cell line OVCAR3 was more sensitive to Etn in vitro than the prostate, breast, and pancreatic cancer cell lines tested. Therefore, Etn, which reduces HIF-1α expression and induces metabolic catastrophe in tumor cells that overexpress CK, may synergize with Nivolumab to offer a direly-needed more efficacious therapy for OCCC.

This is the first study to explore the potential for synergy between an ICI, Nivolumab, and a formulation based on the non-toxic, metabolism-targeting lipid pro-drug, Etn, and the therapeutic efficacy of this combination, for OCCC and more broadly, for EOCs. Etn will reduce HIF-1α expression and selectively target OCCC cells (that intrinsically overexpress CK) by inducing metabolic crisis and altering membrane composition/antigens, which may create favorable conditions for the immunotherapy to be effective. This is the first preclinical study to evaluate absorption, distribution, metabolism, elimination (ADME) and toxicity of Etn-based formulations in a comprehensive manner.

To selectively increase intracellular levels of PhosE in cancer cells, the anticancer activity of Etn was explored. ADM E and pharmacological properties of orally-delivered PhosE and Etn [both phosphatidylethanolamine (PE) lipid precursors] were first compared. Etn displayed better GI tract stability, bioavailability, PK properties and in vitro anticancer activity compared to PhosE. Fortuitously, Etn also lacked CYP-related drug-drug interaction liability. Oral Etn exhibited superior anticancer in vivo efficacy in a prostate cancer xenograft model compared to PhosE. LC/MS showed that higher intracellular PhosE levels correlated with cytotoxicity. Our mechanistic studies identified CK overexpression—a hallmark of metabolic reprogramming in multiple cancer types—in prostate tumor cells compared to adjacent normal. Pharmacological inhibition of CK in prostate cancer cells disrupted conversion of Etn into PhosE, and reduced Etn's cytotoxicity. Analysis of molecular markers revealed that Etn treatment decreased levels of HIF-1α, cell cycle regulators (Cdk2, Cdk4, phosphorylated Rb), and pro-survival molecules (Bcl-2), and increased the levels of p21, Bim, c-PARP, in both cultured (PC-3) cells as well as in PC-3-luc tumors harvested from mice treated orally with Etn. Etn-treated cancer cells showed decreased levels of glucose and glutamine, a reduced OCR, and drastically altered lipid biosynthesis and mitochondrial membrane morphologies indicating pleiotropic effects on metabolic pathways in tumor cells, while sparing normal cells. It was hypothesize that an Etn-based formulation can be developed into a safe, selective, pharmacodynamically- and pharmacokinetically-favorable, IND entity that singly or synergistically with the ICI Nivolumab, provides a novel therapeutic option for chemo-resistant EOCs/OCCC.

Results

Etn exhibits robust and selective antiproliferative activity against a variety of cancer cell lines:

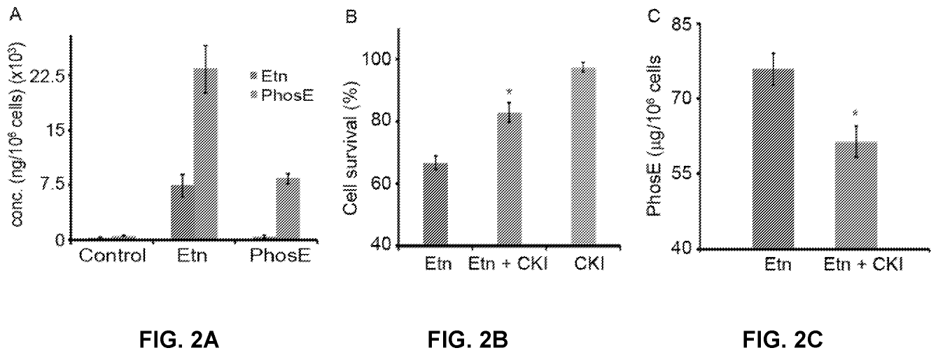

Etn was more effective in inhibiting human prostate PC-3 cell proliferation compared with PhosE (FIG. 1Ai). In a clonogenic assay to assess the reproductive capacity of cells upon drug removal, 2 mg/mL Etn decreased colony numbers by ˜97%; by contrast, 2 mg/mL PhosE was ineffective in decreasing colony numbers (FIG. 1Aii). Moreover, Etn were more effective in reducing viability of prostate cancer lines (PC-3, DU145, and C42B) compared with normal prostate cells (RWPE-1; FIG. 1Bi). To test the generality of Etn's antiproliferative activity on representative cancer cell lines from diverse tissues [breast (MDA-MB-468), ovary (OVCAR-3), colon HCT116-data not shown) and pancreas (CFPAC)], MTT assay was performed to obtain dose-response curves. Etn inhibited proliferation in all the cell lines tested and the ovarian cancer cell line OVCAR3 was the most susceptible to Etn (FIG. 2Bii). PhosE was ineffective in inhibiting proliferation and colony formation of these cell lines up to 100 mg/ml (data not shown).

Inhibition of choline kinase (CK) activity attenuates Etn's antiproliferative activity:

To understand why Etn inhibited cancer cell proliferation more effectively than PhosE, intracellular levels of PhosE and Etn upon treatment with Etn or PhosE were quantified. Both Etn and PhosE treatments increased intracellular PhosE levels but this effect was more pronounced in Etn-treated cells (FIG. 2A); thus, Etn is a pro-drug, which enters tumor cells and gets converted into cytotoxic PhosE. To examine if choline kinase (CK), which is overexpressed in many cancers including prostate and ovarian cancer, catalyzes conversion of Etn into PhosE in PC-3 cells, survival of PC-3 cells upon Etn treatment in the presence/absence of a CK inhibitor was determined. While Etn treatment alone reduced cell proliferation, CK inhibition significantly attenuated Etn's antiproliferative activity (FIG. 2B) and reduced conversion of Etn into PhosE (FIG. 2C).

Etn inhibits tumor growth in a prostate cancer xenograft model:

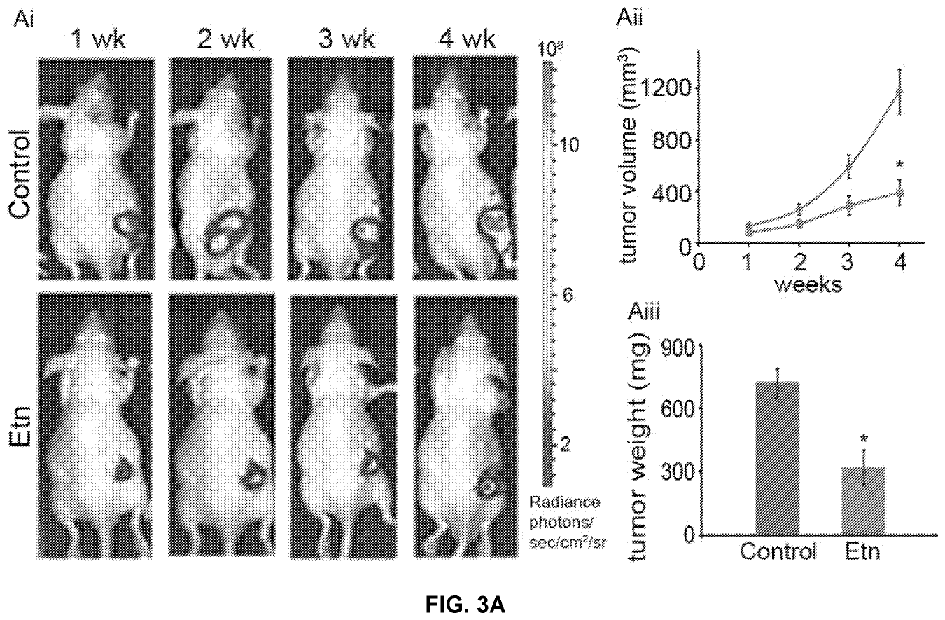

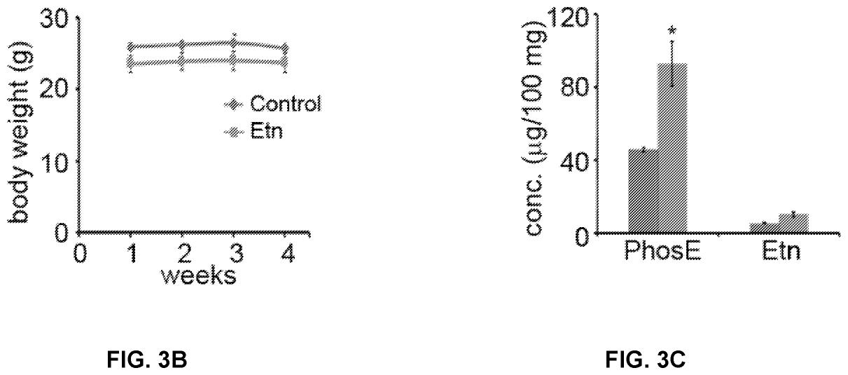

In vivo efficacy of a panel of orally-delivered formulations containing Etn and PhosE Wwase tested in varying molar ratios with pH=5.0 or pH=7.4. Formulations with pH 7.4 and PhosE alone were less effective than Etn in inhibiting tumor growth. Therefore, the formulation with 40 mg/kg Etn, pH=5.0 was pursued. The in vivo efficacy of this formulation was first examined (FIG. 3A). There was an ˜67% reduction in tumor volume (FIG. 3Aii) and ˜55% reduction in tumor weight (FIG. 3Aiii) after 4 weeks of treatment. Importantly, there was no change in body weight of control and Etn-treated mice over this period (FIG. 3B); thus, Etn feeding does not induce any obvious toxicity. Intratumoral PhosE level in Etn-treated mice was ˜38% higher than in controls with no significant change in intratumoral Etn (FIG. 3C).

Etn activates mitochondrially-mediated death pathways in in vitro and in vivo models of prostate cancer:

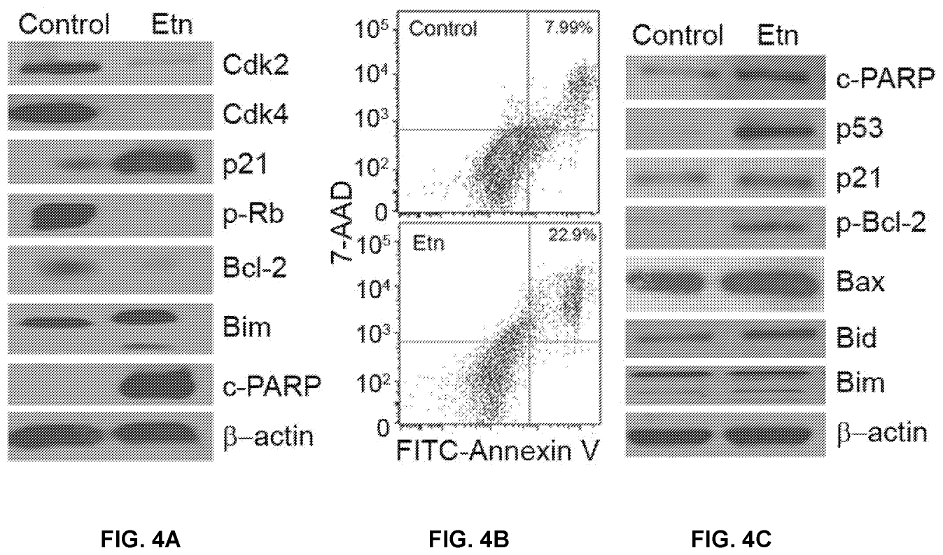

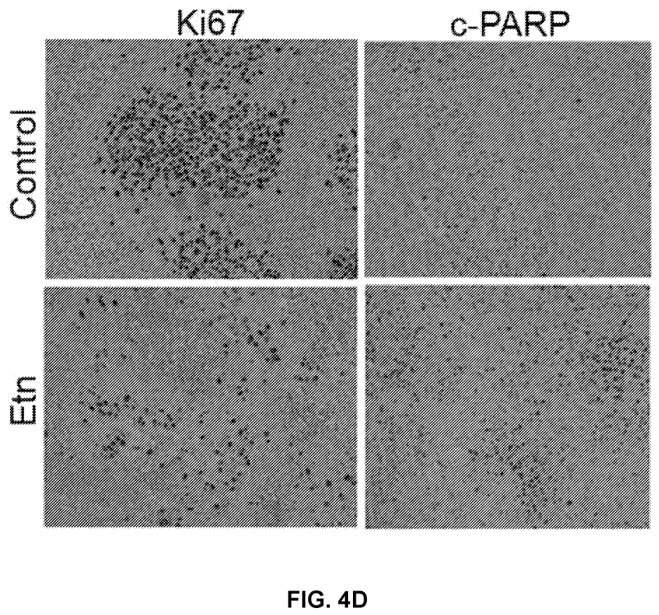

The mechanism of Etn's anticancer activity was then explored in cultured PC-3 cells. Etn treatment downregulated pRb, Cdk4, and Cdk2, and upregulated p21, suggesting that Etn stalls cell cycle progression in PC-3 cells (FIG. 4A). Etn treatment increased levels of proapoptotic markers such as c-PARP and Bim, and decreased antiapoptotic molecules such as Bcl-2, implicating a mitochondrially-mediated death pathway (FIG. 4A). Flow-cytometry was used to show that Etn treatment increased the number of annexin-V positive apoptotic cells (FIG. 4B). Treatment of tumors with 40 mg/kg Etn resulted in upregulation of p53, p21, Bax, pBcl2, c-PARP, Bim and Bid (FIG. 4C), suggesting activation of p53-induced growth arrest and apoptosis. Immunohistochemical staining of FFPE samples for Ki67 (cell proliferation marker) and c-PARP showed marked decrease in Ki67 expression and increase in c-PARP expression in treated tumors in comparison to control tumors (FIG. 4D), confirming that Etn regulates tumor growth by inhibiting cell proliferation and inducing apoptosis.

Etn affects HIF1-α expression and cellular metabolism in in vitro and in vivo models of prostate cancer:

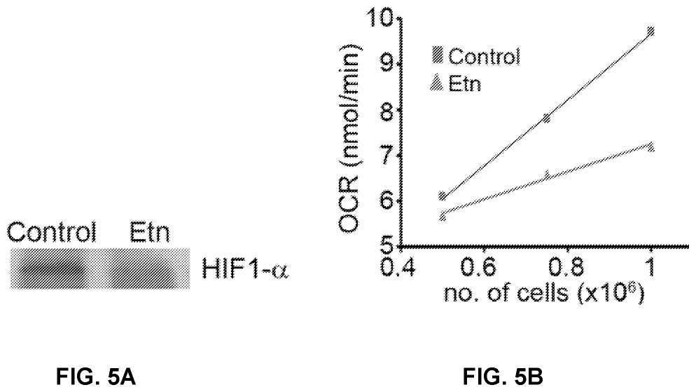

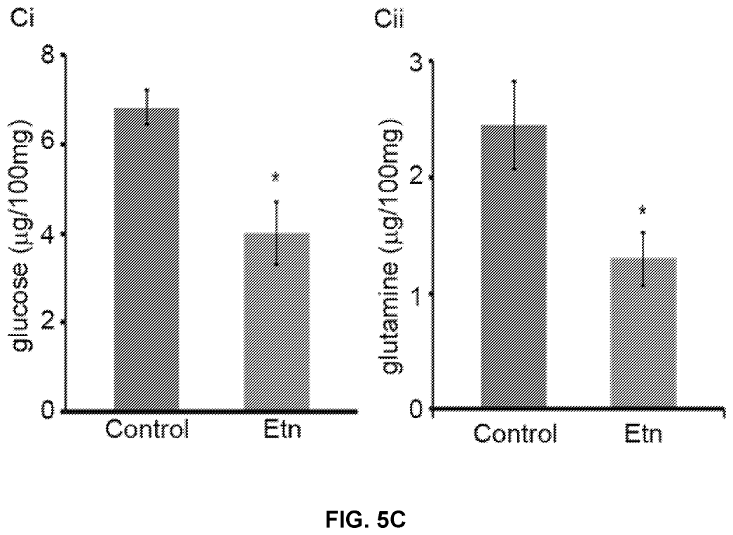

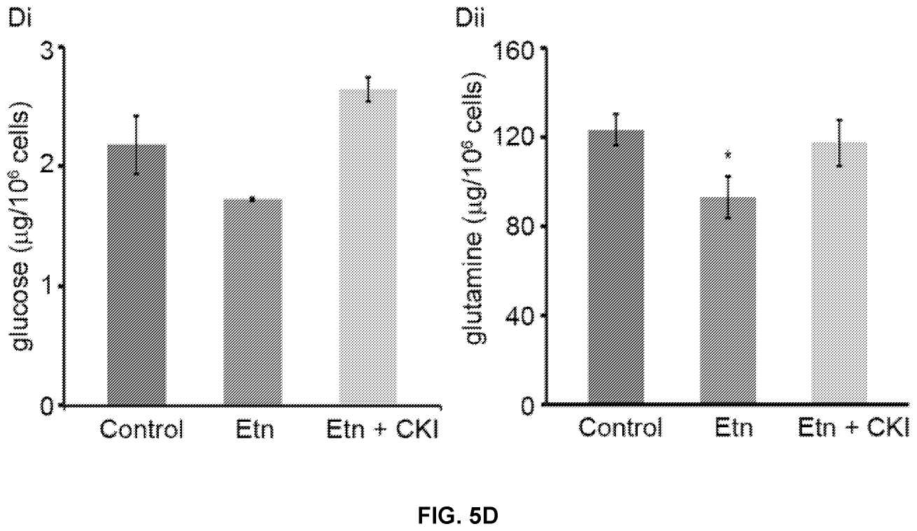

Since p53 is activated upon energetic/metabolic stress in cells, how Etn affects the p53 pathway was examined. It was hypothesized that PhosE accumulation alters HIF1-a expression/function that impairs glucose/glutamine metabolism leading to metabolic stress, which activates p53-induced cell death. Indeed, HIF1-a was strongly downregulated in Etn-treated cells (FIG. 5A). OCR was measured in control and Etn-treated cells, and evaluated the glucose and glutamine content in (a) cultured cells and (b) tumors from control and Etn-treated mice. Etn treatment decreased OCR in PC-3 cells (FIG. 5B). Both glucose and glutamine content were significantly reduced in Etn-treated tumors (FIGS. 5Ci, ii) and cells (FIGS. 5Di, ii), compared to control tumors and cells. Inhibition of CK abrogated the Etn-mediated decrease in cellular glucose and glutamine content (FIGS. 5Di, ii).

Etn alters cellular lipids and impairs mitochondrial integrity in vivo:

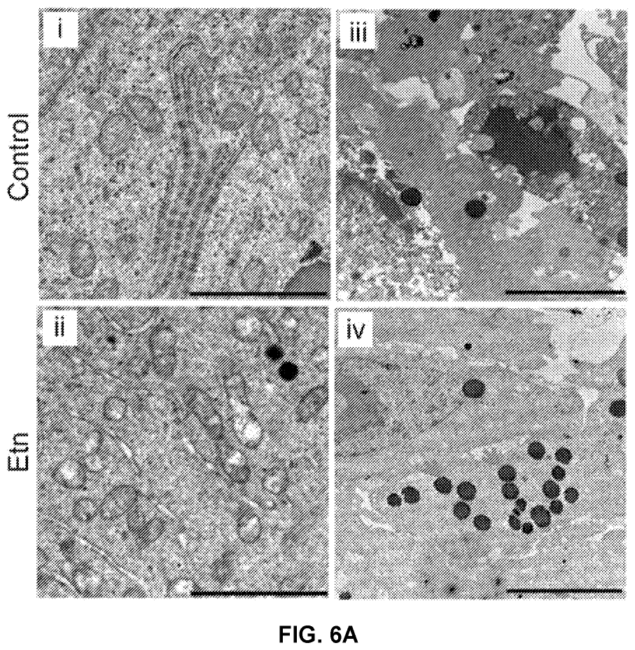

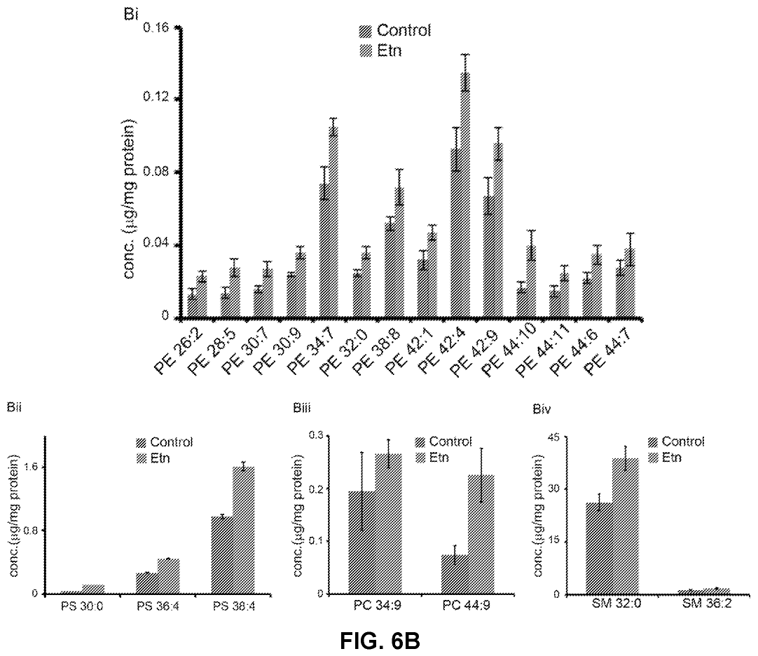

Transmission electron microscopy (TEM) micrographs showed elongated mitochondria with highly degraded matrices in Etn-treated tumors (FIG. 6Aii) compared with controls (FIG. 6Ai). More osmiophilic granules were evident in treated versus control tumors (FIG. 6Aiii, iv); thus, Etn treatment leads to lipid accumulation in cells, alters mitochondrial structure, and likely, induces lipid-mediated activation of cell death pathways. Lipidomic analyses of tumors from control and Etn-treated groups quantified 402 lipids from various lipid classes such as phosphatidylethanolamine (PE), phosphatidylcholine (PC), phosphatidylserine (PS), lysophospholipids, ceramides, and sphingomyelin (SM). Levels of 21 PE lipids (FIG. 6Bi), and other lipids from the PS (FIG. 6Bii), PC (FIG. 6Bii), and SM (FIG. 6Biv) classes were increased in Etn-treated tumors. Thus, PhosE and phospholipid accumulation downregulates HIF-1α, precipitates a bioenergetics/metabolic crisis, activates p53-mediated signaling and culminates in cell death.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating an epithelial ovarian carcinoma (EOC), comprising administering to a subject in need thereof, an effective amount of a first pharmaceutical composition comprising:
monoethanolamine or a pharmaceutically acceptable salt thereof; and
a pharmaceutically effective carrier.

2. The method of claim 1, further comprising administering to the subject an effective amount of a second pharmaceutical composition comprises a checkpoint inhibitor.

3. The method of claim 2, wherein the checkpoint inhibitor comprises an anti-PD-1 antibody, anti-PD-L1 antibody, anti-CTLA-4 antibody, or a combination thereof.

4. The method of claim 3, wherein the checkpoint inhibitor comprises Nivolumab.

5. The method of claim 1, wherein monoethanolamine is the only therapeutically active agent in the first pharmaceutical composition.

6. The method of claim 1, wherein the pharmaceutical composition comprises monoethanolamine and a checkpoint inhibitor.

7. The method of claim 6, wherein the checkpoint inhibitor comprises an anti-PD-1 antibody, anti-PD-L1 antibody, anti-CTLA-4 antibody, or a combination thereof.

8. The method of claim 7, wherein the checkpoint inhibitor comprises Nivolumab.

9. The method of claim 1, wherein the composition is administered by oral, intravenous, intraperitoneal, subcutaneous, intranasal, or dermal administration.

10. The method of claim 1, wherein the composition is administered in capsules.

11. The method of claim 1, wherein the composition is administered as a liquid.

12. The method of claim 11, wherein the composition has a pH value between 2-8.

13. The method of claim 12, wherein the composition has a pH value of about 5.

14. The method of claim 1, wherein the EOC comprises ovarian clear cell carcinoma (OCCC).

15. The method of claim 1, wherein the EOC comprises serous ovarian carcinoma.

16. The method of claim 1, wherein the EOC comprises endometrioid ovarian cancer.

17. The method of claim 1, wherein the EOC comprises mucinous ovarian cancer.

18. A composition comprising monoethanolamine or a pharmaceutically acceptable salt thereof, an anti-PD-1 antibody, and a pharmaceutically effective carrier.
